(19) United States
(12) Patent Application Publication
(10) Pub. No.: US 2018/0125902 A1
(43) Pub. Date: May 10, 2018

(54) USE OF ANTIBIOTICS WITH SPECIFIC THERAPEUTIC ACTIVITIES COMBINED WITH THE SIMULTANEOUS USE OF LACTOBACILLI AND/OR BIFIDOBACTERIA WITH A NON-TRANSFERABLE ANTIBIOTIC RESISTANCE, HAVING THE SAME THERAPEUTIC INDICATION

(71) Applicant: Probiotical S.p.A., Novara (IT)

(72) Inventor: Giovanni Mogna, Novara (IT)

(21) Appl. No.: 15/569,716

(22) PCT Filed: Apr. 29, 2016
(86) PCT No.: PCT/IB2016/052436

§ 371 Date: Oct. 26, 2017

(30) Foreign Application Priority Data
Apr. 30, 2015 (IT) 102015902347522(M

(51) Int. Cl. A61K 35/745; A61P 31/04; A61K 31/395; A61K 35/747; A61K 31/437; A61K 47/36; A61K 9/00

(52) U.S. Cl. CPC A61K 35/745; A61P 31/04; A61K 31/395; A61K 2035/11; A61K 31/437; A61K 47/36; A61K 9/0053; A61K 35/747

(57) ABSTRACT

The present invention relates to the use of antibiotics with specific therapeutic activities combined with the simultaneous use of lactobacilli and/or bifidobacteria having a non-transferable antibiotic resistance and with the same therapeutic indication as said antibiotics. In particular, the present invention relates to a composition comprising or, alternatively, consisting of antibiotics suitable for the treatment of acute and chronic intestinal infections due to gram-positive and gram-negative bacteria; diarrheic syndromes; diarrhea due to an imbalance of the intestinal microflora such as for example, summer diarrhea, traveler's diarrhea and enterocolitis; pre- and postoperative prophylaxis of infectious complications in gastrointestinal surgery; gastrointestinal diseases such as irritable bowel syndrome (IBS), constipation, alterations of intestinal microflora; chronic inflammatory intestinal diseases (IBD) and inflammations due to Helicobacter pylori; said antibiotics belonging to the rifamycin family, preferably rifaximin, being combined with specific strains of bacteria belonging to the species Bifidobacterium longum having the same therapeutic indication as said antibiotics.

### CROSS-REFERENCE TO RELATED APPLICATIONS

This application is a national phase application of PCT Application No. PCT/IB2016/052436, filed Apr. 29, 2016, which claims the benefit of Italian Application No. MI2015A000624, filed Apr. 30, 2015, which are incorporated by reference herein in their entireties and for all purposes.

### DETAILED DESCRIPTION OF THE INVENTION

The present invention relates to the use of antibiotics with specific therapeutic activities combined with the simultaneous use of lactobacilli and/or bifidobacteria with a non-transferable antibiotic resistance and having the same therapeutic indication as said antibiotics. In particular, the present invention relates to a composition comprising or, alternatively, consisting of antibiotics suitable for the use in the treatment of acute and chronic intestinal infections due to gram-positive and gram-negative bacteria; diarrheic syndromes; diarrhea due to an imbalance in the intestinal microflora such as for example, summer diarrhea, traveler's diarrhea and enterocolitis; pre- and postoperative prophylaxis of infectious complications in gastrointestinal surgery; gastrointestinal diseases such as irritable bowel syndrome (IBS), constipation and alterations of intestinal microflora, chronic inflammatory intestinal diseases (CIID) (also known as inflammatory bowel diseases (IBD)) which comprise Crohn's disease and ulcerative rectocolitis; said antibiotics being combined with specific strains of bacteria belonging to the species Bifidobacterium longum having the same therapeutic indication as said antibiotics (simultaneous or concurrent administration).

Taking antibiotics, which are useful for fighting and eradicating bacterial infections for example in the event of acute and chronic gastrointestinal infections, diarrheic syndromes, irritable bowel syndrome (IBS), chronic inflammatory intestinal diseases (IBD) and infections by Helicobacter pylori, often leads to serious side effects to the body, in particular towards the bacterial flora of intestine and mucosae. Antibiotics are also prescribed as a preoperative prophylaxis before surgical or dental procedures. The intended aim, by using antibiotics, is to prevent the proliferation of pathogenic bacteria, which can cause dangerous, even systemic, infections. There exist targeted antibiotics, those which only suppress some specific families of pathogenic bacteria, as well as those “with a broad spectrum activity”, being suitable when the body is subjected to infections of unclear etiology, or as a preventive measure.

One of the known unwanted side effects, related to the use of antibiotics, is due to the fact that besides to kill pathogenic bacteria, which are responsible for bacterial infections, antibiotics also destroy the indigenous “good” bacterial flora which colonizes both intestine and mucosae giving rise to, among others, the following symptoms such as: colitis, irritable bowel syndrome (IBS) and intestinal inflammatory disorders characterized by diarrhea, feces in the form of separate hard lumps with occurrence of mucus, abdominal swelling and cramps; Candida albicans infections which can affect both intimate (typically, vaginal candidiasis in women) and buccal mucosae with the development of whitish plaques and symptoms such as burning and redness; stomatitis and occurrence of oral aphthae; nausea and stomachache, as well as a weakened immune system resulting in a body more vulnerable to additional infections.

The irritable bowel syndrome (IBS), which, from a clinical point of view, is characterized by the coexistence of abdominal pain and/or swelling as well as alteration of bowels, is often related to an imbalance among the different species of intestinal bacteria. Bowel alterations can be represented by diarrhea, constipation, or both.

In order to limit the adverse effects on the “good” bacterial flora due to antibiotics, it is recommended to take, along with antibiotics, chemical substances belonging to the group of gastro-protective agents which, however, are not devoid of limits and drawbacks or the postponed, not simultaneous, intake of probiotic bacteria at least 8-12 hours after starting antibiotics, in order to prevent antibiotics from also acting against said probiotic bacteria leading to a useless administration of the latter. However, taking probiotic bacteria at least 8-12 hours after the administration of antibiotics is also unable to avoid that a subsequent intake of antibiotics would cause their destruction or inactivation and, thus, in any case prevents them from colonizing the intestinal tract.

Nevertheless, it would be very useful to be able to administrate at the same time, for a specific and well-defined bacterial infection to be treated, an antibiotic having a suitable therapeutic indication effective against said infection, and strains of probiotic bacteria (simultaneous administration) with activity for the same therapeutic indication as the antibiotics being used, in order to preventing or minimizing the effects from an imbalance of the indigenous “good” bacterial flora which colonizes both intestine and mucosae, resulting from the use of antibiotics, thereby assuring a predominance of indigenous bacterial species relative to pathogens so that to avoid all the adverse effects resulting from the antibiotic intake.

The Applicant, following to a long and intense research and development activity, solved the above cited limits and drawbacks of the know art.

Basically, the Applicant found useful to administering a combination consisting of selected antibiotics with a specific and established therapeutic activity against a particular and well-determined infection or disease/disorder to be treated, in association with selected strains of bacteria having a proved antibiotic resistance non-transferable to other species, and effectively used in the treatment for the same therapeutic indication as the antibiotics themselves.

It is an object of the present invention a composition comprising or, alternatively, consisting of antibiotics, used for specific therapeutic indications, in association with strains of lactobacilli and/or bifidobacteria; said lactobacilli and/or bifidobacteria having a proved antibiotic resistance non-transferable to other species, and being further used for the same therapeutic indications as said antibiotics; said composition being for use in the curative treatment of:

(i) acute and chronic intestinal infections due to gram-positive and gram-negative bacteria;

(ii) diarrheic syndromes;

(iii) diarrhea due to an imbalance of the intestinal microflora selected from summer diarrhea, traveler's diarrhea and enterocolitis;

(iv) pre- and postoperative prophylaxis of infectious complications in gastrointestinal surgery;

(v) gastrointestinal diseases such as irritable bowel syndrome (IBS), constipation and alterations of intestinal microflora;

(vi) chronic inflammatory intestinal diseases (CIID) (also known as inflammatory bowel diseases (IBD)) comprising Crohn's disease and ulcerative rectocolitis;

(vii) infections by Helicobacter pylori.

Preferred embodiments of the present invention will be evident from the following detailed description.

Within the context of the present invention by “compositions of the present invention” is meant food or nutraceutical compositions, or supplement product compositions or medical device compositions (even for oral use), or pharmaceutical compositions.

It is an object of the present invention selected strains of bacteria belonging to the species Bifidobacterium longum, for use in the treatment of gastrointestinal diseases such as irritable bowel syndrome (IBS), diarrhea, constipation, alterations of intestinal microflora, chronic inflammatory intestinal diseases (IBD) and infections by Helicobacter pylori, in association with antibiotics, as claimed in the appended claims.

Within the context of the present invention by “association” is meant that the antibiotic, selected from the group comprising or, alternatively, consisting of rifamycins, and the strain of bacteria belonging to the species Bifidobacterium longum can be concurrently administered since they can be thoroughly mixed together within the same pharmaceutical formulation, such as for example in a tablet, or hard gel capsule, or packet, or stick, or oil. However, the present invention also encompasses the concept of “association” in which the antibiotic, selected from the group comprising or, alternatively, consisting of rifamycins, and the strain of bacteria belonging to the species Bifidobacterium longum can be individually administered, since they are not physically together in the same pharmaceutical formulation, but within a short period of time from each other. For example, the antibiotic can be administered in the form of a tablet (or different pharmaceutical forms) and, shortly thereafter, the bacterial strain can be presented in the form of water-dispersible granules, or capsule, or tablet, or packet, or stick, or oil.

It is an object of the present invention food or nutraceutical compositions, or supplement product compositions, or medical device compositions (even for oral use), comprising a mixture of bacteria which comprises or, alternatively, consists of said strains of bacteria belonging to the species Bifidobacterium longum, for use in the treatment of gastrointestinal diseases such as irritable bowel syndrome (IBS), diarrhea, constipation, alterations of intestinal microflora, chronic inflammatory intestinal diseases (IBD) and inflammations due to Helicobacter pylori, as claimed in the appended claims. Said compositions being used in association with antibiotics.

It is an object of the present invention pharmaceutical compositions comprising a mixture of bacteria which comprises or, alternatively, consists of said strains of bacteria belonging to the species Bifidobacterium longum and antibiotics selected from rifamycins (in particular, rifaximin), for use in the treatment of gastrointestinal diseases such as irritable bowel syndrome (IBS), diarrhea, constipation, alterations of intestinal microflora, chronic inflammatory intestinal diseases (IBD) and inflammations due to Helicobacter pylori, as claimed in the appended claims.

The Applicant investigated and selected a number of strains of bacteria belonging to various bacterial species and in particular belonging to the species Bifidobacterium longum. Following to an extended research activity, the Applicant achieved to select, among others, the strain of bacteria belonging to the species Bifidobacterium longum W11, deposited at the Belgian Coordinated Collection of Microorganisms—BCCM LMG, with access number LMG P-21586.

The present invention relates to biologically pure cultures of the strain of bacteria Bifidobacterium longum W11 LMG P-21586 and the use thereof as probiotic for preparing a pharmaceutical composition further comprising, in association, an antibiotic selected from rifamycins (in particular rifaximin), for use in the curative treatment of infections, or syndromes, or diseases, or disorders as described in the above cited items from (i) to (vii); advantageously for use in the treatment of gastrointestinal diseases such as irritable bowel syndrome (IBS), diarrhea, constipation, alterations of intestinal microflora, chronic inflammatory intestinal diseases (IBD) and inflammations due to Helicobacter pylori.

The strain of bacteria Bifidobacterium longum W11 LMG P-21586 was selected because, besides to show a capability to colonizing the intestinal bacterial flora and adhering to the intestinal cells—characteristics which all render them optimal probiotic agents able to promote a good health of the gastrointestinal tract and restore the functionality impaired by the use of antibiotics—said strain exhibits a surprising and unexpected antibiotic resistance which was shown to be unexpectedly non-transferable to other species. Furthermore, said strain is advantageously suitable for the use in the treatment of gastrointestinal diseases such as irritable bowel syndrome (IBS), diarrhea, constipation, alterations of intestinal microflora, chronic inflammatory intestinal diseases (IBD) and inflammations due to Helicobacter pylori.

The Applicant carried out a complete sequencing of the overall genome of the strain of bacteria Bifidobacterium longum W11 LMG P-21586 and further conducted a comparative genomic analysis. The results allowed to identify a genetic locus related to the resistance to the antibiotic rifamycin and semisynthetic derivatives thereof such as rifaximin, rifampicin, rifabutin and rifapentine. The genetic locus is located on the gene rpoB encoding the RNA polymerase beta required for all the transcriptional processes of the bacterium. The analysis of the plasmid DNA revealed the absence of genes involved in rifamycin resistance. Therefore, a locus transferability, which confers resistance to the antibiotic rifamycin cannot occur. Furthermore, the absence of DNA elements indicating a genetic modification of the bacterial strain was observed.

The antibiotic resistance shown by the above cited strain, object of the present invention, was studied and observed towards rifamycin and semisynthetic derivatives thereof such as rifaximin, rifampicin, rifabutin and rifapentine. The antibiotic resistance of a bacterial strain belonging to the genus Lactobacillus and Bifidobacterium, and its non-transferability to other species, along with the ability of said strain to be used for the same therapeutic indication as the antibiotic, collectively represent the fundamental and required characteristics which the strain should possess in order to be selected and used within the context of the present invention. The Applicant further tested and confirmed the non-transferability of said antibiotic resistance in the plasmid content as well.

Therefore, for the first time, the strain of bacteria of the present invention can be formulated along with natural active substances or synthetic or semisynthetic chemical molecules, which all have antibiotic activity, to give a pharmaceutical composition which can be effectively administered, in a safe and advantageous manner, to patients in order to treating and curing bacterial infections without causing side effects typical of treatments with antibiotics alone.

The chemical active substances or natural molecules with antibiotic activity (briefly, antibiotics) are selected from the group comprising or, alternatively, consisting of the family of rifamycins derived from Streptomices mediterranei, or synthetic or semisynthetic chemical molecules. Rifamycins usually have a broad-spectrum antibacterial activity against Gram+, Gram− bacteria and mycobacteria and, furthermore, a mechanism of action based on the inhibition of the RNA-nucleotidyltransferase (DNA-dependent RNA-polymerase) by forming very stable complexes 1:1 with said enzyme. Rifamycins generally have a very low toxicity, are poorly absorbed by oral route and are mainly excreted through the bile. The semisynthetic derivatives of rifamycin are selected from rifaximin, rifampicin, rifabutin and rifapentine.

Bacterial cultures of the strain of bacteria Bifidobacterium longum W11 LMG P-21586 were firstly produced, according to techniques and apparatuses known to the skilled in the field, in the laboratory and then as industrial preparations. Basically, pure strains were grown at about 37° C. for approximately 16 hours in a culture medium TPY based on casein peptone, yeast extract, glucose and mineral salts. From these primary cultures, subsequent subcultures were prepared, in order to increase the number of cells of the starting pure strains until to obtain stock cultures, which were subsequently used as inoculum for industrially producing a bacterial culture of the strain of bacteria Bifidobacterium longum W 11 LMG P-21586.

Bacterial cultures thus prepared contain on average from about 1×10⁹ to 1×10¹² live cells of Bifidobacterium longum W11 LMG P-21586 per gram, as analyzed according to a method based on a flow cytofluorimetric technique, known to the skilled in the field.

Bacterial cultures are known to be tested in a colonization test in the mouse intestine with positive results since the count of the bacterial load of Bifidobacterium longum W 11 LMG P-21586 shown a value greater than 1×10⁶ live cells per gram of mouse feces.

The efficacy of the bacterial culture and, consequently, also the efficacy of the composition containing said bacterial culture in favoring the gastrointestinal health and in the treatment of intestinal diseases such as diarrhea, traveler's diarrhea, gastroenteritis, constipation, irritable colon, diverticulitis and intestinal inflammatory diseases can be enhanced by adding to said composition food prebiotic fibers in the form of non-digestible oligosaccharides such as, for example, fructooligosaccharides known as FOS, or inulin, or galactooligosaccharides known as GOS, or xylooligosaccharides known as XOS, or arabinoxylooligosaccharides known as AXOS, which are neither absorbed nor hydrolyzed in the first intestinal tract and enhance the activity as well as stimulate the metabolism of the strain of bacteria Bifidobacterium longum W11 LMG P-21586 to the detriment of pathogenic bacteria.

Preferred oligosaccharides are mixtures of fructooligosaccharides consisting of a basic unit of a glucose (G) molecule bound to a strand of fructose (F) molecules with general formula GFn, n being less than or equal to 4, having a polymerization degree, namely, a number of monosaccharide units, within 2 and 20. Fructooligosaccharides, which are effectively used in the compositions of the present invention, have a polymerization degree comprised from 2 to 10.

The compositions of the present invention, besides to contain an antibiotic of the rifamycin family and the strain of bacteria Bifidobacterium longum W11 LMG P-21586, can comprise several excipients such as sweeteners, for example mannitol, aspartame or sorbitol, flavoring and coloring agents, vitamins, preferably B-group vitamins such as vitamin B1, B2, B6, B9 and B12 and/or D-group vitamins such as vitamin D2 and D3. B-group vitamins are aimed to further support the subject's health by restoring the vitamin component, which is strongly reduced during gastrointestinal diseases and further impaired by the use of antibiotics.

The compositions of the present invention preferably consist of compositions which can be orally administered in the form of capsules, tablets, granules, compressed lozenges and oil, all containing a bacterial load of live and viable cells, due to the sophisticated production technology being used, which is comprised, at the end of the stability period of 24 months, from 1×10⁶ to 1×10″, preferably from 1×10⁷ to 1×10⁹.

The strain of bacteria Bifidobacterium longum W11 LMG P-21586 is produced by the Applicant through a technology which allows the cells of the strain to be encapsulated in a protein matrix, forming protein microspheres which contain in their inside the encapsulated cells, making them more resistant both to the gastroduodenal transit and stress affecting the cells during the end product storage. The protein coating prevents from applying the common microbiological techniques for bacterial counting used to assess the bacterial count of a culture. This is because the protein coating is insoluble in diluents and reagents used in the above cited microbiological techniques for bacterial counting, impeding to prepare 10 serial dilutions of the freeze-dried bacterial culture being required. Furthermore, said protein microspheres can encapsulate a variable number of cells (from few to thousands of cells), but by said count only the number of microspheres is determined, making impossible to obtain the effective number of cells. From the above reasons, the Applicant developed a method, which uses a flow cytofluorimetric technique (Internal method 615).

The Applicant carried out some stability tests, by using the internal method 615, in a sample C of the strain of bacteria Bifidobacterium longum W11 LMG P-21586 with cells coated with a protein matrix at +25° C. The results reported in Table 1 surprisingly show almost no mortality, after 9 months at 25° C.

TABLE 1

| | Results | Results | Results | Results |
|---|---|---|---|---|
| Sample | T0 months | T3 months | T6 months | T9 months |
| C | 42 × 10⁹ | 42 × 10⁹ | 41 × 10⁹ CFU/g | 40 × 10⁹ CFU/g |
| | CFU/g | CFU/g | | |

The tests for assessing the bacterial count showed that the strain of bacteria Bifidobacterium longum W11 LMG P-21586 is resistant to such an extent that it does not undergo a significant mortality, both during its storage and during the technological processes for producing the compositions of the present invention. In fact, these bacterial compositions were subjected to stability tests which demonstrated an optimal resistance of the strain of bacteria Bifidobacterium longum W11 LMG P-21586 at room temperature of 25° C. so that to allow the marketing thereof up to 24 months from the strain production.

The Applicant conducted further tests concerning the osmotic stress stability (extreme conditions of osmotic stress) in sterile water at +25° C. at t0 and t7 (after 7 days) in a first sample C1 of viable cells of the strain of bacteria Bifidobacterium longum W 11 LMG P-21586 coated with a protein matrix (gastro-protected) and a second sample C2 of viable cells of the strain of bacteria Bifidobacterium longum W 11 LMG P-21586 (non gastro-protected). The results of the reduction are shown in Table 2.

TABLE 2

| | | | Results t7 | % |
|---|---|---|---|---|
| Sample | Test method | Results t0 | (days) | Mortality |
| C1 | Internal | 105 × 10⁹ cell/g | 103 × 10⁹ cell/g | 2% |
| | method 612 | | | |
| C2 | Internal | 12 × 10⁹ cell/g | 5 × 10⁹ cell/g | 62% |
| | method 612 | | | |

The compositions of the present invention can further contain prebiotic fibers such as fructooligosaccharides known as FOS, or inulin, or galactooligosaccharides known as GOS, or xylooligosaccharides known as XOS, or arabinoxylooligosaccharides known as AXOS, preferably from 0.5 g to 5 g of fructooligosaccharides FOS, or galactooligosaccharides GOS, per dose, even more preferably from 1 g to 3 g, and/or vitamins, preferably B-group vitamins such as vitamin B1, B2, B6, B9 and B12 and/or D-group vitamins such as vitamin D2 and D3.

An embodiment of the present invention comprises:
the strain of bacteria Bifidobacterium longum W11 LMG P-21586 coated with a protein matrix which allows them to overcome the barrier of gastric juices and bile acids, thus reaching the intestine with a high activity (live and viable cells for a favorable replication),
a prebiotic fiber selected from fructooligosaccharides, such as the product commercially known as Actilight® or inulin, or galactooligosaccharides GOS, or xylooligosaccharides XOS, or arabinoxylooligosaccharides AXOS, which is able to cross intact both the stomach and small intestine in order to reach the colon where it becomes a specific nutrient for the Bifidobacteria growth (bifidogenic activity),
one or more vitamins of group B and/or group D,
an antibiotic belonging to the group of rifamycins, in particular rifaximin,
pharmaceutically acceptable excipients, flavors and stabilizers.

With reference to the antibiotics used in the present invention mention can be made, for example, of rifamycin in the form of 200 mg film-coated tablets or granules for oral suspension, 2 g/100 ml (for example Normix®).

1-7. (canceled)

8. A composition comprising
(a) a rifamycin family antibiotic; and
(b) Bifidobacterium longum W 11 deposited as LMG P-21586 at the Belgian Coordinated Collection of Microorganisms—BCCM LMG, wherein the Bifidobacterium has a non-transferable resistance to the rifamycin family antibiotic.

9. The composition of claim 8, wherein the Bifidobacterium longum W 11 is coated in a protein matrix.

10. The composition of claim 8, wherein the Bifidobacterium longum W11 is present in a concentration of 1×10⁹-1×10¹² live cells per gram.

11. The composition of claim 8, wherein the composition further comprises Lactobacilli.

12. The composition of claim 8, wherein the rifamycin family antibiotic is selected from the group consisting of rifamycin, rifaximin, rifampicin, rifabutin and rifapentine.

13. The composition of claim 8, wherein the rifamycin family antibiotic is rifaximin.

14. The composition of claim 8, wherein the composition further comprises a prebiotic fiber selected from fructooligosaccharides, inulin, galactooligosaccharides, xylooligosaccharides, and arabinoxylooligosaccharides.

15. The composition of claim 8, wherein the composition further comprises one or more B-group vitamins, one or more D-group vitamins or both.

16. The composition of claim 8, wherein the composition comprises:
(a) Bifidobacterium longum W11 in a concentration of 1×10⁹-1×10¹² live cells per gram;
(b) rifaximin;
(c) a fructooligosacchardie or a galactooligosaccharide;
(d) one or more B-group vitamins, one or more D-group vitamins or both; and
(e) a pharmaceutically-acceptable excipient.

17. A method for treating in a subject an indication selected from the group consisting of a gram-positive intestinal infection, a gram-negative intestinal infection, a diarrheic syndromes, summer diarrhea, traveler's diarrhea, enterocolitis, preoperative prophylaxis of infectious complications in gastrointestinal surgery, postoperative prophylaxis of infectious complications in gastrointestinal surgery, irritable bowel syndrome (IBS), constipation, intestinal microflora alterations, Crohn's disease, ulcerative rectocolitis, and Helicobacter pylori infection, comprising orally administering to the subject the composition of claim 8.

18. A method for treating in a subject an indication selected from the group consisting of a gram-positive intestinal infection, a gram-negative intestinal infection, a diarrheic syndromes, summer diarrhea, traveler's diarrhea, enterocolitis, preoperative prophylaxis of infectious complications in gastrointestinal surgery, postoperative prophylaxis of infectious complications in gastrointestinal surgery, irritable bowel syndrome (IBS), constipation, intestinal microflora alterations, Crohn's disease, ulcerative rectocolitis, and Helicobacter pylori infection, comprising administering to the subject:
(a) a rifamycin family antibiotic; and
(b) Bifidobacterium longum W 11 deposited as LMG P-21586 at the Belgian Coordinated Collection of Microorganisms—BCCM LMG, wherein the Bifidobacterium has a non-transferable resistance to the rifamycin family antibiotic.

19. The method of claim 18, wherein the rifamycin family antibiotic and the Bifidobacterium longum W11 are orally administered.

20. The method of claim 18, wherein the rifamycin family antibiotic and the Bifidobacterium longum W 11 are administered in separate formulations.

21. The method of claim 18, wherein the rifamycin family antibiotic and the Bifidobacterium longum W 11 are administered in the same formulation.

22. The method of claim 18, wherein the rifamycin family antibiotic is selected from the group consisting of rifamycin, rifaximin, rifampicin, rifabutin and rifapentine.

23. The method of claim 18, wherein the rifamycin family antibiotic is rifaximin.

24. The method of claim 18, wherein the composition further comprises Lactobacilli.

25. The method of claim 18, wherein the subject is administered a composition comprising the Bifidobacterium longum W11 in a concentration of 1×10⁹-1×10¹² live cells per gram.

26. The method of claim 25, wherein the Bifidobacterium longum W 11 is coated in a protein matrix.

27. The method of claim 25, wherein the composition further comprises a prebiotic fiber selected from fructooligosaccharides, inulin, galactooligosaccharides, xylooligosaccharides, and arabinoxylooligosaccharides.

28. The method of claim 18, wherein the subject is further administered one or more B-group vitamins, one or more D-group vitamins or both.
